(19) United States
(12) Patent Application Publication
(10) Pub. No.: US 2007/0218562 A1
(43) Pub. Date: Sep. 20, 2007

(54) Color indicator for halamine treated fabric

(76) Inventors: Shulong Li, Spartanburg, SC (US); Ling Li, Spartanburg, SC (US); Jack W. Spoon, Spartanburg, SC (US)

Correspondence Address: Legal Department (M-495), P.O. Box 1926, Spartanburg, SC, 29304, US

(21) Appl. No.: 11/384,620

(22) Filed: Mar. 20, 2006

(51) Int. Cl. G01N 33/00

(52) U.S. Cl. 436/106

(57) ABSTRACT

The invention relates to a halamine compound indicator comprising a halamine compound treated substrate and a redox color indicating composition disposed on a selected area of the substrate, wherein the redox color indicator comprises a compound capable of reacting with the halamine compound and exhibiting color change as a result of the reaction. Methods of using the halamine compound indicator are also covered.

### FIELD OF THE INVENTION

The present invention refers to a color indicator for halamine treated fabric which easily tests for the presence of halamine compounds on substrates and textiles.

### BACKGROUND

There is growing concern over emerging infectious deceases and bacterial growth. It is well known to place antimicrobial agents, such as quaternary ammonium salts, phosphonium salts, halamine compounds, and silver based compounds, on substrates and textiles to reduce or prevent bacterial growth. Halamine compounds have been shown to be effective antimicrobial agents on textile and substrates to reduce or prevent bacterial growth. However, antimicrobial agents are only effective when they are present in an effective amount on the substrate or textile, when first purchased and after laundering a number of times.

In the past, textiles have been tested for halamine compounds by cutting a section of fabric and testing for chlorine using a headspace test as described in U.S. Pat. No. 6,835,865 or stirring the fabric for a certain time in a potassium iodide/acid solution, then titrating the solution with a standard sodium thiosulfate solution with starch indicator. These methods determined the amount of halamine on textiles, but the tests destroy at least a section of the textile, require expensive equipment, and take a significant amount of time and training to perform.

There is a need for a consumer or commercial laundry service to be able to easily and quickly tell if a substrate or textile has or still has an effective amount of halamine compound.

### DETAILED DESCRIPTION

In the present invention, the halamine compound indicator is used to determine if a substrate has been treated with a halamine compound particularly, if a substrate has significant amount of halamine for antimicrobial effect. In order to test for the presence of a halamine composition on a substrate or fabric, a redox color indicator is applied to a selected area of the substrate or textile. The redox color indicator reacts with the halamine compound (if present) and changes color. The user can tell that if there is no color change, there is little or no halamine compound on the substrate or textile. If there is a color change where the redox color indicator was applied, the user can tell that there is an antimicrobial effective amount of halamine on the substrate or textile.

The halamine compound treated substrate is a textile material comprising a plurality of yarns, the yarns comprising a halamine compound disposed on the exterior surface of the yarns and/or dispersed in the interior portion of the yarns.

The treated substrate can be any suitable textile material. The textile material can comprise a plurality of yarns provided in a knit or woven construction, or the textile material can comprise a plurality of fibers that are provided in a non-woven construction. The yarns or fibers from which the textile material is constructed can contain natural fibers, synthetic fibers, or any suitable combination thereof. Suitable fibers include, but are not limited to, cellulose (e.g., cotton and rayon), polyamides, polyesters, polyethylenes, polypropylenes, polyacrylics, cellulose acetate, polylactic acid, silk, wool, glass, polyaramids, and combinations thereof. In a preferred embodiment, the textile material comprises yarns comprising fibers selected from the group consisting of cellulose fibers (e.g., cotton and rayon), polyester fibers, and combinations thereof (e.g., blends of cotton and polyester fibers).

As utilized herein, the term “halamine” refers to an amine, amide, imide, urea, melamine, sulfonamide, hydantoin, urethane, and their derivatives in which a hydrogen attached to the nitrogen atom has been replaced with a halogen atom (e.g., a chlorine atom). The halamine compound can have any suitable molecular weight. Typically, the halamine compound has a molecular weight of about 2,500 atomic mass units or less, preferably about 2,000 atomic mass units or less, or about 1,000 atomic mass units or less, or about 500 atomic mass units or less. Any known halamine compound may be used. Such compounds are disclosed, for example, in U.S. application Ser. No. 11/256,887 which is incorporated herein, in its entirety, by reference. Other halamine compounds have been disclosed in U.S. Pat. No. 6,077,319 which is also incorporated herein by reference.

Redox color indicating compounds are those materials that can undergo a redox reaction with a halamine compound and exhibit color change as a result of such reaction. Halamine compounds, in general, are strong oxidizing agents. The redox indicator agent contains a reducing agent, and a chromophore which exhibits change in light absorption, reflection and/or fluorescence as a result of the redox reaction between a halamine compound and a reducing agent causing the change in color. As utilized herein, “color change” refers to change in light absorption, reflection, or fluorescence which can be easily observed visually or with the help of a simple instrument. Herein, “light” refers to radiation in ultraviolet, visible, near infrared and infrared wavelength ranges. In one embodiment, a fluorescence dye (such as optical brighteners including fluorescence compounds based on stilbene derivatives, pyrazolines, styryl derivatives of benzene and biphenyl, bis(benzoxazol-2-yl) derivatives, coumarins, and carbostyryls) is used as a redox color indicator agent. The oxidative halamine reacts with such fluorescence dye to alter or to destroy the fluorescence effect. Such color change is thus observed by placing the indicator applied substrate under a black light. Lack of fluorescence or change in fluorescence indicates halamine presence on the substrate. “Color change” in visible, ultraviolet, and infrared ranges can also be easily visualized or detected by using a hand-hold photometer, calorimeter, a digital camera, a lens fitted with a filter, and other similar light detecting instruments known to an ordinary skill in the art.

The redox color indicating composition comprises at least one redox agent that reacts with a halamine compound to produce a detectable color change. Redox agents that may be used include, but are not limited to iodide salts, iodide/starch mixtures, o-toluidine, N,N-diethyl-p-phenylene diamine, neutral red, safranin T, phenosafranin, indigomono sulphonic acid, indigocarmine, indigotrisulphonic acid, indigotetrasuphonic acid, methylene blue, thionine, sodium o-cresol indophenol, sodium 2,6-dichlorphenol indophenol, diphenylene, diphenylbenzidine, sodium diphenylamine sulfonate, o-dianisidine, n-ethoxylchrysoidine, n-phenylanthranilic acid, and mixtures thereof.

In one embodiment, the redox agent is a colorless mixture comprising a salt of iodide and starch (herein after referred to as the “I Agent”). Iodide reacts with halamine compounds to form iodine and starch forms a complex with iodine which exhibits a yellow to blue or brown color. Since iodide and starch are both nontoxic and inexpensive, such mixture as color indicating redox agent is especially suitable for detecting and verifying the presence of antimicrobial halamine on a consumer product. The I Agent is washable, meaning that the color washes off of substrates and textiles after reacting and forming a color on the substrate or textile and therefore will not permanently stain an article. The I Agent may be used in a concentration of between 0.1 and 10%, preferably between 0.5% to 1% by weight of the redox color indicating solution. It has been found that this concentration provides a strong color change and easy wash-off from textiles in a subsequent laundry. It has also been found that color change depends on concentration of iodide salt and starch in the mixture and concentration of halamine. Therefore, different concentrations of iodide and starch mixture can be used to detect halamine at different concentrations. In a preferred embodiment, a solution containing concentration about 0.5% iodide and 5 ppm starch (optional) is used to detect halamines in 30-500 ppm amount on a substrate.

In one embodiment, the redox agent is an o-dianisidine, preferably used as an o-dianisidine acidic aqueous solution, herein after referred to as the “D Agent”. Its chemical structure is found below.

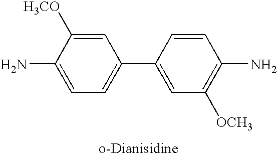

The D Agent reacts with halamine compounds to for a form a pink to red coloration. D Agent may be used in a concentration of between 0.005 and 1%, preferably 0.05 and 0.5% by weight of the redox color indicating solution. It has been found that the color change depends on concentration of the D Agent in the mixture and concentration of halamine. Therefore, different concentrations of the D Agent can be used to detect halamine at different concentrations. In a preferred embodiment, a solution containing concentration about 0.1% by weight D Agent with a pH of 1 may is used to detect halamines in 30-500 ppm amount on a substrate.

In another embodiment, the redox agent is an o-toluidine, preferably used as an o-toluidone acidic aqueous solution, herein after referred to as the “T Agent”. Its chemical structure is found below.

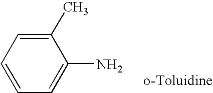

The T Agent reacts with halamine compounds to form a blue to yellow color. The T Agent may be used in a concentration of between 0.005 and 1%, preferably 0.05 to 0.5% by weight of the redox color indicating solution. It has been found that the color change of the T Agent depends on concentration of T Agent in the mixture and concentration of halamine on the textile. Therefore, different concentrations of T Agent can be used to detect halamine at different concentrations. In a preferred embodiment, a solution containing concentration about 0.1% by weight T Agent at a pH of 1 may be used to detect halamine compounds in 30-500 ppm amount on a substrate.

In one embodiment, the redox color indicating solution comprises a redox agent, water, and an acid. The acid may be, but not limited to acetic acid, hydrochloric acid, sulfuric acid, nitric acid, formic acid, proprionic acid, oxalic acid, and citric acid. The solution of the redox agent, water, and acid will typically have a pH of between about 1 and 6. Such a pH range facilitates the redox reaction between the redox indicator composition and a halamine compound.

The halamine may be applied to the substrate or textile by any suitable method. For example, the halamine compound can be applied to a package of yarn(s) then formed into a textile. The textile material can also be produced by treating a textile material with a halamine compound using conventional spray, dip coating, or impregnation processes. The processes can employ one or more of elevated temperatures, fiber swelling solvents, dye carriers, plasticizers, steam (e.g., both atmospheric steam and super-heated steam), infrared radiation, sonication, supercritical fluids (e.g., supercritical carbon dioxide), etc. Typically, elevated temperatures (e.g., temperatures of about 100° C. to about 200° C.) and/or steam are used in treating the textile material. When the halamine compound is applied using a foam application, spray, dip coating, or impregnation process, the treated fabric typically is dried in an oven at a temperature of about 150° C. to about 200° C. for about 30 seconds to about 10 minutes. More details of halamine and treating textiles with halamines may be found in co-pending application Ser. No. 11/256,887.

Alternatively, halamine compound is applied to the substrate by first treating a substrate with a halamine precursor such as organic compound having at least one of amine, urea, melamine, urethane, amide, sulfonamide, imide, and hydantoin moieties, and subsequently contacting the halamine precursor treated substrate to a halogenated solution such as chlorine bleach. Embodiments of such method of forming halamine treated substrate have been described in U.S. application Ser. No. 11/256,887 and in U.S. Pat. No. 6,077,319 which are incorporated herein, in its entirety, by reference.

The redox color indicating composition is applied to a selected region of the substrate and if a halamine compound is present, the redox color indicating composition will change color (typically from clear to yellow, red, or blue). If there is no halamine compound in the selected area tested, the redox color indicating composition will remain the same color or remain colorless. In one embodiment, the redox color indicating composition is applied using a liquid dropper. In another embodiment, the redox color indicating composition is applied using a marker device, such as a marker pen with the redox coloring indicating solution as the ink. These methods are preferred because they are able to deliver the redox color indicating composition to a selected region of the fabric. A consumer or industrial laundering service may use a liquid dropper or marker to test an inconspicuous area of the substrate or fabric (such as an inside seam of a drape or garment) for the presence of halamine. In one embodiment, there may be a tab of fabric sewn into a seam for testing. The test method is a quick, easy, inexpensive, and nondestructive test to ensure that the antimicrobial nature of the garment or drape is still in place. Other marking devices to apply such inventive redox color indicator composition, including wet wipe, tape, printer, sprayer, brush, and similar devices known to an ordinary skill in the art, are also conceived.

In yet another embodiment, the redox color indicator composition is applied to a textile permanently or semi-permanently by a coating, impregnation, printing, dyeing, or finishing process. When an antimicrobially effective amount of halamine forms on a textile, by exposing to a chlorine bleach solution for example, the textile treated with redox color indicator composition will show a color change, indicating the presence of halamine compound. When a reducing agent is subsequently applied to the fabric, the redox color indicator composition is returned to the original color, indicating the removal or lack of halamine compound.

In one embodiment, after the redox color indicator composition is applied to a substrate and color change is observed, an additional reducing agent is further applied to the selected area of the substrate to, lessening or negating the color change of the redox agent. This additional step removes any appearance alteration or staining as a result of the application of a redox color indicating composition. In one embodiment, reducing agent may include, but is not limited to, bisulfites, sulfides, thiosulfates, antioxidants such as vitamins including ascorbic acid, hydroborides, hydroazine, thiourea, and phenols. The amount of the reducing agent should be sufficient to return the redox color indication composition to the original reduced form and to react fully with the halamine in the selected area of the substrate. Alternatively, the color marking on a treated halamine fabric can be lessened or removed by a subsequent laundry cycle.

The redox color indicator composition can also be used to detect the presence of physically adsorbed chlorine gas, hypochlorite, perchlorate, iodine, periodate, perbromate, and similar oxidative halogenated species that is capable of reacting with redox color indicator composition. Cotton fibers and fabrics, for example, are prone to retain chlorine bleach used in a laundry process by physically adsorption. The redox color indicator composition can be used to detect the presence of physically adsorbed chlorine on a cotton fiber containing fabric.

### EXAMPLES

Halamine Treated and Control Fabrics

The examples used 4 types of fabrics; 100% cotton fabric, cotton/polyester (35/65 blend) woven fabric, 100% polyester woven fabric, and polyester nonwoven fabric. Untreated samples of each fabric were used for controls.

The halamine solution to treat the fabrics was formed from 2 grams of Cyasorb®UV-3346 (available from Cytec Industries Inc.), 1 gram of acetic acid and 0.5 gram of Akrophobe DAN (available from Clariant Corporation) in 100 milliliters of deionized water. The fabrics were individually dipped in the halamine solution bath and padded at 40 psi nip pressure. The fabrics were dried at 360° F. (182° C.) for 5 minutes. Finally, the finished fabrics were washed in a home washing machine, followed by a rinse cycle with a solution containing 0.1% Clorox® regular bleach solution. Then, the fabrics were dried for 30 minutes in a home drying machine to give halamine treated fabrics.

### Example 1

### “I Agent” Marker

Example 1 was the preparation of the I Agent, formation of the I Agent Marker, and testing of the I Agent Marker.

The I Agent was prepared by mixing 0.50 gram of potassium iodide (KI), 0.05 milliliters of acetic acid and 0.05 milliliters of 1% soluble starch aqueous solution, and diluting the solution to 100 milliliters with deionized water. An absorbent ink tube of the marker was immersed into the I Agent solution for 10 minutes to saturate the absorbent ink tube. The absorbent ink tube was inserted into a plastic marker jacket to form an I Agent Marker.

Both the untreated control fabrics and halamine treated fabrics were tested with the I Agent Marker by contacting the fabric with the I Agent Marker pen tip and sliding the marker tip cross a selected area on the fabrics. Table 1 summaries the color results of testing the I Agent Marker on the halamine treated and control fabrics.

TABLE 1
Color change of I Agent Marker on halamine treated textiles and
untreated textiles

| | Control | Halamine Treated |
|---|---|---|
| 100% cotton fabric, and | No color change | Brown |
| cotton/polyester (35/65 blend) | No color change | Brown |
| woven fabric, | | |
| 100% polyester woven fabric, | No color change | Yellow |
| 100% polyester nonwoven fabric | No color change | Yellow |

As can be seen from Table 1, the untreated control fabrics did not show any color change at the I Agent Maker applied area. The halamine treated fabrics showed a color change to yellow or brown at the I Agent Marker applied area. The color change to yellow or brown can show the relative amounts of halamine on the fabric with the darker brown the color change, the more halamine on the treated fabric. The color at the I Agent Marker applied area can be lessened or negated by washing or the application of a reducing agent.

### Example 2

### “D Agent” Marker

Example 2 was the preparation of the D Agent, formation of the D Agent Marker, and testing of the D Agent Marker.

The D Agent was prepared by mixing 0.10 gram of o-dianisidine, and 1.0 milliliters of acetic acid, and then diluting the solution to 100 milliliters with deionized water. An absorbent ink tube of a marker was immersed into the I Agent solution for 10 minutes to saturate the absorbent ink tube. The absorbent ink tube was inserted into a plastic marker jacket to form a D Agent Marker.

Both the untreated control fabrics and halamine treated fabrics were tested with the D Agent Marker by contacting the fabric with the D Agent Marker pen tip and sliding the marker tip cross a selected area on the fabrics. Table 2 summaries the color results of testing the D Agent Marker on the halamine treated and control fabrics.

TABLE 2
Color change of D Agent Marker on halamine treated textiles and
untreated textiles

| | Control | Halamine Treated |
|---|---|---|
| 100% cotton fabric, and | No color change | Red |
| cotton/polyester (35/65 blend) | No color change | Red |
| woven fabric, | | |
| 100% polyester woven fabric, | No color change | Pink |
| 100% polyester nonwoven fabric | No color change | Pink |

As can be seen from Table 2, the untreated control fabrics did not show any color change at the D Maker applied area. The halamine treated fabrics showed a color change to pink or red at the D Agent Marker applied area. The color change to pink or red depends on the amount of the halamine treatment on the fabrics, the more red the resultant color is, the more halamine is present on the textile. The color at the D Agent Marker applied area can be lessened or negated by further washing or the application of a reducing agent.

### Example 3

### “T Agent” Marker

Example 3 was the preparation of the T Agent, formation of the T Agent Marker, and testing of the T Agent Marker.

The ink solution for the T agent marker was prepared by mixing 0.10 gram of o-toluidine and 1.0 milliliters of acetic acid, and then diluting the solution to 100 milliliters with deionized water. An absorbent ink tube of a marker was immersed into the T Agent solution for 10 minutes to saturate the absorbent ink tube. The absorbent ink tube was inserted into a plastic marker jacket to form a T Agent Marker.

Both the untreated control fabrics and halamine treated fabrics were tested with the T Agent Marker by contacting the fabric with the T Agent Marker pen tip and sliding the marker tip cross a selected area on the fabrics. Table 3 summaries the color results of testing the T Agent Marker on the halamine treated and control fabrics.

TABLE 3
Color change of T Agent Marker on halamine treated textiles and
untreated textiles

| | Control | Halamine Treated |
|---|---|---|
| 100% cotton fabric, and | No color change | Yellow |
| cotton/polyester (35/65 blend) | No color change | Yellow |
| woven fabric, | | |
| 100% polyester woven fabric, | No color change | Light Blue |
| 100% polyester nonwoven fabric | No color change | Light Blue |

As can be seen from Table 3, the untreated control fabrics did not show any color change at the T Maker applied area. The halamine treated fabrics showed a color change to blue or yellow at the T Marker applied area. The color change to blue or yellow depends on the amount of the halamine treatment on the fabrics. The color at the T Marker applied area can be lessened or negated by further washing or the application of a reducing agent.

It is intended that the scope of the present invention include all modifications that incorporate its principal design features, and that the scope and limitations of the present invention are to be determined by the scope of the appended claims and their equivalents. It also should be understood, therefore, that the inventive concepts herein described are interchangeable and/or they can be used together in still other permutations of the present invention, and that other modifications and substitutions will be apparent to those skilled in the art from the foregoing description of the preferred embodiments without departing from the spirit or scope of the present invention.

What is claimed is:

1. A halamine compound indicator comprising a halamine compound treated substrate and a redox color indicating composition disposed on a selected area of the substrate, wherein the redox color indicator comprises a compound capable of reacting with the halamine compound and exhibiting color change as a result of the reaction.

2. The halamine compound indicator of claim 1, wherein the substrate is a textile.

3. The halamine compound indicator of claim 1, wherein said halamine compound is an organic compound having a moiety selected from the group consisting of hydantoin, melamine, urea, urethane, amide, imide, amine, sulfonamide, and the combination thereof.

4. The halamine compound indicator of claim 3, wherein the halamine compound has a molecular weight of about 2000 atomic mass units or less and is selected from the group consisting one of the following structures (I)-(V) 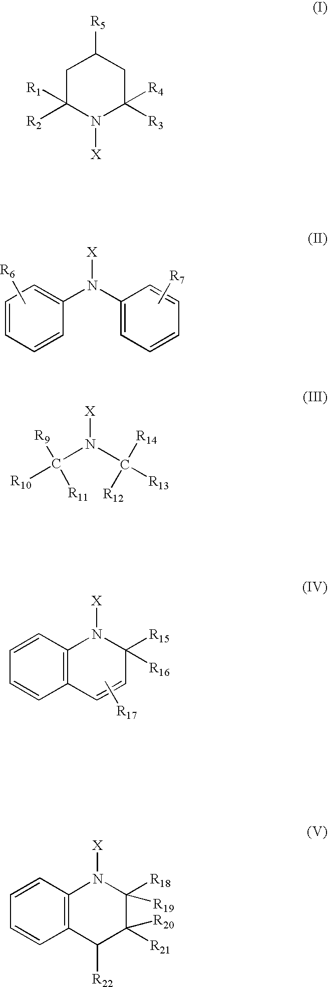
wherein R₁, R₂, R₃, and R₄ are independently selected from the group consisting of C₁-C₄ alkyl groups; R₅ is selected from the group consisting of a hydrogen atom, an alkyl group, an alkyl amine group, a cyclic amine group, an amide group, a cyclic amide group, an isocyanate group, a hydroxyl group, an ether group, an ester group, and combinations thereof; R₆ and R₇ are independently selected from the group consisting of a hydrogen atom, an alkyl group, an aryl group, an amine group, an amide group, and combinations thereof; R₉, R₁₁, R₁₂, and R₁₄ are independently selected from the group consisting of C₁-C₄ alkyl groups; R₁₀ and R₁₃ are independently selected from the group consisting of an alkyl group, an aryl group, an amine group, an amide group, and combinations thereof; R,₅ and R₁₆ are independently selected from the group consisting of C₁-C₄ alkyl groups; R₁₇ is selected from the group consisting of a hydrogen atom, an alkyl group, an aryl group, an amine group, an amide group, and combinations thereof; R₁₈, R₁₉, R₂₀, and R₂₁ are independently selected from the group consisting of C₁-C₄ alkyl groups; R₂₂ is selected from the group consisting of a hydrogen atom, an alkyl group, an aryl group, an amine group, an amide group, and combinations thereof; and X a chlorine atom, a bromine atom, or an iodine atom.

5. The halamine compound indicator of claim 1, wherein the redox color indicating composition comprises at least one redox agent selected from the group consisting of iodide salts, iodide/starch mixtures, o-tolidine, N,N-diethyl-p-phenylene diamine, neutral red, safranin T, phenosafranin, indigomono sulphonic acid, indigocarmine, indigotrisulphonic acid, indigotetrasuphonic acid, methylene blue, thionine, sodium o-cresol indophenol, sodium 2,6-dichlorphenol indophenol, diphenylene, diphenylbenzidine, sodium diphenylamine sulfonate, o-dianisidine, n-ethoxylchrysoidine, n-phenylanthranilic acid, and mixtures thereof.

6. The halamine compound indicator of claim 5, wherein the redox color indicating composition comprises a redox agent, water, and an acid.

7. The halamine compound indicator of claim 6, wherein the acid is selected form the group consisting of acetic acid, hydrochloric acid, sulfuric acid, nitric acid, formic acid, proprionic acid, oxalic acid, and citric acid.

8. The halamine compound indicator of claim 6, wherein the pH of the redox color indicating solution has a pH of between 1 and 6.

9. The halamine compound indicator of claim 5, wherein the redox agent comprises an o-dianisidine.

10. The halamine compound indicator of claim 9, wherein the o-dianisidine is in an amount of between 0.005 and 1% by weight of the redox color indicating solution.

11. The halamine compound indicator of claim 5, wherein the redox agent comprises a mixture of a salt of iodide and starch.

12. The halamine compound indicator of claim 11, wherein the mixture of a salt of iodide and starch is in an amount of between 0.1 and 10% by weight of the redox color indicating solution.

13. The halamine compound indicator of claim 5, wherein the redox agent comprises an o-toluidine.

14. The halamine compound indicator of claim 13, wherein the o-toluidine is in an amount of between 0.005 and 1% by weight of the redox color indicating solution.

15. A process for detecting halamine compounds comprising in order:
providing a substrate;
forming a halamine compound on the substrate;
applying a redox color indicating composition to a selected region of the substrate; and,
observing the selected region of the material for a color change.

16. The process of claim 15, wherein the substrate is a textile.

17. The process of claim 15, wherein the halamine compound has a molecular weight of about 2000 atomic mass units or less and is selected from the group consisting of one of the following structures (I)-(V) 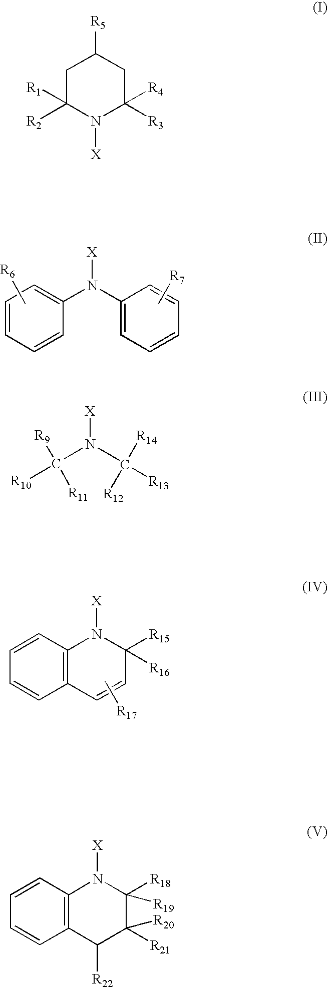
wherein R₁, R₂, R₃, and R4 are independently selected from the group consisting of C₁-C₄ alkyl groups; R₅ is selected from the group consisting of a hydrogen atom, an alkyl group, an alkyl amine group, a cyclic amine group, an amide group, a cyclic amide group, an isocyanate group, a hydroxyl group, an ether group, an ester group, and combinations thereof; R₆ and R₇ are independently selected from the group consisting of a hydrogen atom, an alkyl group, an aryl group, an amine group, an amide group, and combinations thereof; R₉, R₁₁, R₁₂, and R₁₄ are independently selected from the group consisting of C₁-C₄ alkyl groups; R₁₀ and R₁₃ are independently selected from the group consisting of an alkyl group, an aryl group, an amine group, an amide group, and combinations thereof; R₁₅ and R₁₆ are independently selected from the group consisting of C₁-C₄ alkyl groups; R₁₇ is selected from the group consisting of a hydrogen atom, an alkyl group, an aryl group, an amine group, an amide group, and combinations thereof; R₁₈, R₁₉, R₂₀, and R₂₁ are independently selected from the group consisting of C₁-C₄ alkyl groups; R₂₂ is selected from the group consisting of a hydrogen atom, an alkyl group, an aryl group, an amine group, an amide group, and combinations thereof; and X is a chlorine atom, a bromine atom, or an iodine atom.

18. The process of claim 15, wherein the redox color indicating composition comprises a redox agent, water, and an acid.

19. The process of claim 15, wherein the redox color indictaing composition comprises at least one redox agent selected from the group consisting of iodide salts, iodide/starch mixtures, o-tolidine, N,N-diethyl-p-phenylene diamine, neutral red, safranin T, phenosafranin, indigomono sulphonic acid, indigocarmine, indigotrisulphonic acid, indigotetrasuphonic acid, methylene blue, thionine, sodium o-cresol indophenol, sodium 2,6-dichlorphenol indophenol, diphenylene, diphenylbenzidine, sodium diphenylamine sulfonate, o-dianisidine, n-ethoxylchrysoidine, n-phenylanthranilic acid, and mixtures thereof.

20. The process of claim 15, further comprising applying a reducing agent to the selected region of the substrate.

21. The process of claim 20, wherein the reducing agent is selected from the group of salts consisting of bisulfite, sulfide, thiosulfate, hydroboride, hydroazine, thiourea, and phenols.

22. The process of claim 15, wherein the redox color indicating composition is applied to a selected region of the substrate using a liquid dropper.

23. The process of claim 15, wherein the redox color indicating solution is applied to a selected region of the substrate using a marker device.

24. The process of claim 16, wherein detecting halamine compounds is nondestructive to the textile.
